(19) United States
(12) Patent Application Publication
(10) Pub. No.: US 2025/0250381 A1
(43) Pub. Date: Aug. 7, 2025

(54) METHODS OF SYNTHESIZING DIALYKYLAMINO DISULFIDE DYNAMIC CROSSLINKERS

(71) Applicants: Braskem America, Inc., Philadelphia, PA (US); Northwestern University, Evanston, IL (US)

(72) Inventors: John Mark Torkelson, Skokie, IL (US); Tapas Debsharma, Evanston, IL (US); Nathan Suryajaya Purwanto, Morton Grove, IL (US); Sarah Mitchell, Pittsburgh, PA (US); Kimberly Miller McLoughlin, Pittsburgh, PA (US); Jayme Kennedy, Pittsburgh, PA (US)

(21) Appl. No.: 19/046,839

(22) Filed: Feb. 6, 2025

Related U.S. Application Data
(60) Provisional application No. 63/550,236, filed on Feb. 6, 2024.

(51) Int. Cl. C08F 246/00; C07D 211/96

(52) U.S. Cl. CPC C08F 246/00; C07D 211/96; C08F 2810/20

(57) ABSTRACT

Provided are methods of synthesizing polymerizable alkylamino disulfide compounds which may be used as dynamic crosslinkers to form dynamic crosslinked polymer networks.

### PRIORITY CLAIM

This application claims priority under 35 U.S.C. § 119(e) to U.S. Patent Application No. 63/550,236, filed Feb. 6, 2025, which is herein incorporated by reference in its entirety.

### BACKGROUND

Relative to thermoplastics, traditional thermoset polymers with three-dimensional cross-linked networks exhibit superior dimensional stability at the expense of malleability.¹ The presence of percolated permanent covalent cross-links makes conventional thermosets non-reshapeable, nonrecyclable, and non-repairable. Due to this inherent unrecyclability of thermosets, they typically meet their end-of-life fate in the form of landfilling, incineration, or as devalued filler materials. To tackle this issue of unrecyclability, a new class of polymer materials called covalent adaptable networks (CANs) was introduced, providing both cross-linked structures at use conditions and malleability owing to their external stimuli-based reversible covalent bonds. A range of reversible or dynamic covalent bonds, such as esters, silyl ethers, siloxanes, amides, and β-amino esters, among others, have been used in CANs.

### SUMMARY

Provided are methods of synthesizing polymerizable alkylamino disulfide compounds which may be used as dynamic crosslinkers to form dynamic crosslinked polymer networks (a type of covalent adaptable network (CAN)). The present disclosure is based on the inventors' understanding of limitations of existing synthetic techniques and application of modifications which result in improved dynamic crosslinkers and thus, improved reprocessability of dynamic crosslinked polymer networks formed therefrom. As demonstrated in the Example, below, unexpectedly faster elevated-temperature stress relaxation times were observed from the dynamic crosslinker bis(2,2,6,6-tetramethyl-4-piperidyl methacrylate) disulfide (BiTEMPS methacrylate or BTMA) formed using the present methods as compared to the same dynamic crosslinker formed using an existing synthetic technique.

Specifically, the Example below describes a modified synthesis for BTMA that achieves significantly higher yield, higher purity, and less color, with essentially exclusively disulfide linkages compared to BTMA produced by an existing synthetic technique with relatively low yield, low purity, and more color with a mixture of oligosulfide linkages. Herein, BTMA synthesized using the present methods (and CANs formed therefrom) may be referenced using the term “present” while BTMA synthesized using existing synthetic techniques (and CANs formed therefrom) may be referenced using the term “comparative.” Low levels (5 mol %) of present BTMA were used to form CANs with n-hexyl methacrylate (present BTMA-HMA CANs). The present BTMA-HMA CANs maintain their cross-link densities after reprocessing. Additionally, compared with comparative BTMA-HMA CANs formed from comparative BTMA, the present BTMA-HMA CANs exhibit significantly faster stress relaxation at elevated temperatures, which manifests in rapid reprocessability. Remarkably, a present BTMA-HMA CAN formed using 5 mol % BTMA requires only 5 min at 130° C. by compression molding for reprocessing with full cross-link density recovery. Significantly, this is a factor of four faster than comparative BTMA CANs formed using comparative BTMA, offering the potential for rapid processing of CAN materials.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIG. 1. A) Synthesis of comparative BTMA (mixture of BTMA-S2, BTMA-S3, BTMA-S4) using an existing synthetic technique (top) and present BTMA (BTMA-S2) (bottom). B) Optimization of present BTMA synthesis by varying solvent and temperature. C) BTMA-HMA CAN synthesis by free radical polymerization using 5 mol % BTMA.

FIG. 2. Liquid Chromatography (LC) data for the A) present BTMA (BTMA-S2), B) comparative BTMA, and C) blank (methanol), D) mass spectrometry (MS) of protonated TMPM (Scheme S1) and BTMAs with different sulfide linkages.

FIG. 3. A unit cell of a random crystal from present BTMA measured in single-crystal X-ray crystallography showing two BTMA molecules with exclusively disulfide linkages. Grey, red, purple, and yellow represent carbon, oxygen, nitrogen, and sulfur atoms, respectively. Hydrogen atoms are omitted for clarity.

FIG. 4. A) DMA comparison of 1^{st} mold samples of comparative and present BTMA-HMA CAN materials. DMA comparison of B) comparative BTMA-HMA CAN materials and C) present BTMA-HMA CAN materials as a function of remolding. Based on Flory's theory of ideal rubbery elasticity,³⁹ the excellent reproduction of the storage modulus in the quasi-rubbery plateau region (as well as at all studied temperatures) indicates the full recovery of cross-link density after reprocessing.

FIG. 5. A) Normalized stress-relaxation data of comparative BTMA-HMA CAN at various temperatures. B) Normalized stress-relaxation data of present BTMA-PHMA at various temperatures. C) Arrhenius-type plots relating the natural logarithm of average relaxation time to inverse temperature and yielding apparent stress-relaxation activation energies of comparative and present BTMA-HMA CANs.

FIG. 6. Cross-link density recovery of the samples reprocessed by compression molding for different time scales, as assessed by recovery of storage modulus in the quasi-rubbery plateau region: A) comparative BTMA-HMA CAN and B) present BTMA-HMA CAN.

FIG. 7. Structures of illustrative polymerizable alkylamines.

FIG. S1 depicts a ¹HNMR of crude new BTMA synthesized in dry DCM at −70° C.

FIG. S2 depicts a TGA of old and new BTMA.

FIG. S3 depicts a unit cell of a random crystal from old BTMA measured in single-crystal X-ray crystallography. Grey, red, purple, and yellow represent carbon, oxygen, nitrogen, and sulfur atoms, respectively.

FIG. S4 depicts a DSC thermogram of different BTMA-PHMA materials.

FIG. S5 depicts a UV-Vis diffuse reflectance spectroscopy of old and new BTMA-PHMA materials.

FIG. S6 depicts images of compression-molded samples of new and old BTMA-PHMA samples at different reprocessing times under 10 MPa and 130° C.

FIG. S7 depicts the cross-link density of the samples reprocessed at different times plotted in log scale. The graph on the left (a) shows old BTMA-PHMA, and the graph on the right (b) shows new BTMA-PHMA.

FIG. S8 depicts the recovery of cross-link density following 5 min preprocessing compression molding at 130° C.

### DETAILED DESCRIPTION

Provided are methods of synthesizing dialkylamino disulfide dynamic crosslinkers. In an embodiment, such a method comprises forming a reaction mixture by combining a polymerizable alkylamine with an anhydrous source of sulfur in an anhydrous solvent under an anhydrous atmosphere to form a reaction product comprising (or consisting of) a dialkylamino disulfide dynamic crosslinker; and recovering the reaction product from the reaction mixture. The term “anhydrous” means free of water. Due to the inherent nature of chemical synthesis, free does not require a perfect absence of water in the source of sulfur, solvent, or atmosphere. The anhydrous nature of the sulfur source, solvent, and atmosphere is further described below.

The polymerizable alkylamine is an alkylamine having a polymerizable group covalently bound thereto. The polymerizable alkylamine may be represented by PG-(R₁)(R₂)NH, wherein PG is the polymerizable group; and wherein R₁ and R₂ are each an alkyl group (independently selected) and “-” represents a covalent bond to either R₁ or R₂ (e.g., a carbon atom thereof), or wherein R₁ and R₂ are covalently bound together to form a cycloalkyl group containing the nitrogen atom and “-” represents a covalent bond to the cycloalkyl group (e.g., to a carbon atom thereof). The alkyl group may be a linear alkyl group or a branched alkyl group. The alkyl group may have from 1 to 15 carbon atoms. This includes 1, 2, 3, 4, 5, 6, 7, 8, 9, 10, 11, 12, 13, 14, or 15 carbon atoms. The alkyl group may contain no heteroatoms (i.e., only carbon and hydrogen are present). Illustrative alkyl groups include methyl, ethyl, propyl, butyl, pentyl, heptyl, tert-butyl, and 3-methylbutyl. The cycloalkyl group may have from 4 to 15 carbon atoms. This includes 4, 5, 6, 7, 8, 9, 10, 11, 12, 13, 14, or 15 carbon atoms. The cycloalkyl group may be that which is formed by any two of the disclosed alkyl groups (one as R₁ and the other as R₂) being covalently bound together to form the cycloalkyl group containing the nitrogen atom. Other than the nitrogen atom, the cycloalkyl group may contain no heteroatoms (i.e., only carbon and hydrogen are present).

The polymerizable group (PG) covalently bound to the alkylamine comprises a carbon-carbon double bond capable of undergoing free radical polymerization. Illustrative polymerizable groups include vinyl groups, vinylidene groups, and allyl groups. In embodiments, the polymerizable group is a (meth)acrylate group, i.e., H₂CCR₃C(O)O—, wherein “-” represents the covalent bond to the alkylamine and R₃ is hydrogen or methyl. As noted above, this covalent bond may be to a carbon atom of either R₁ or R₂ or, if R₁ and R₂ are covalently bound to together to form a cycloalkyl group containing the nitrogen atom, to a carbon atom of the cycloalkyl group.

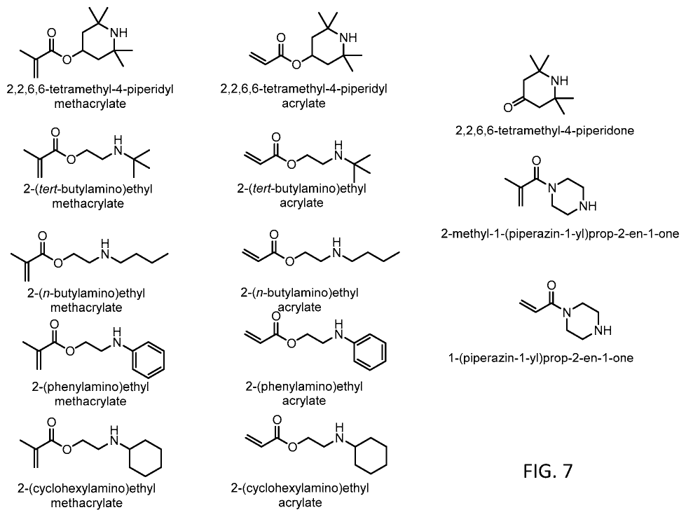

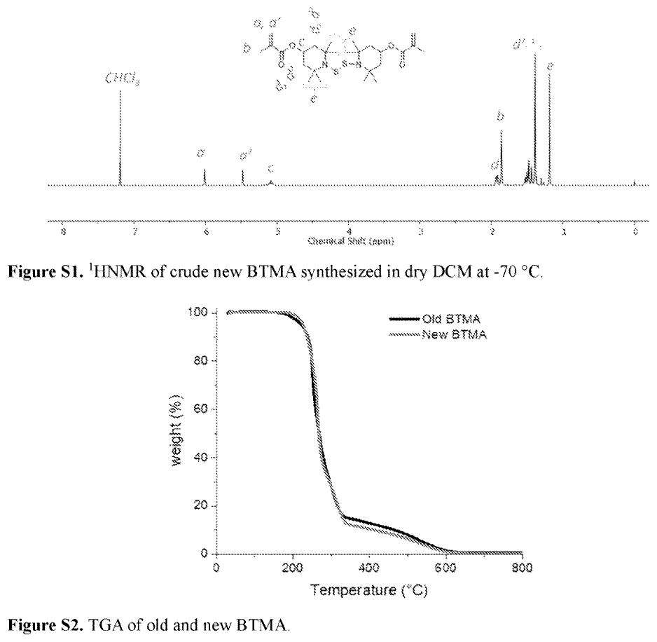

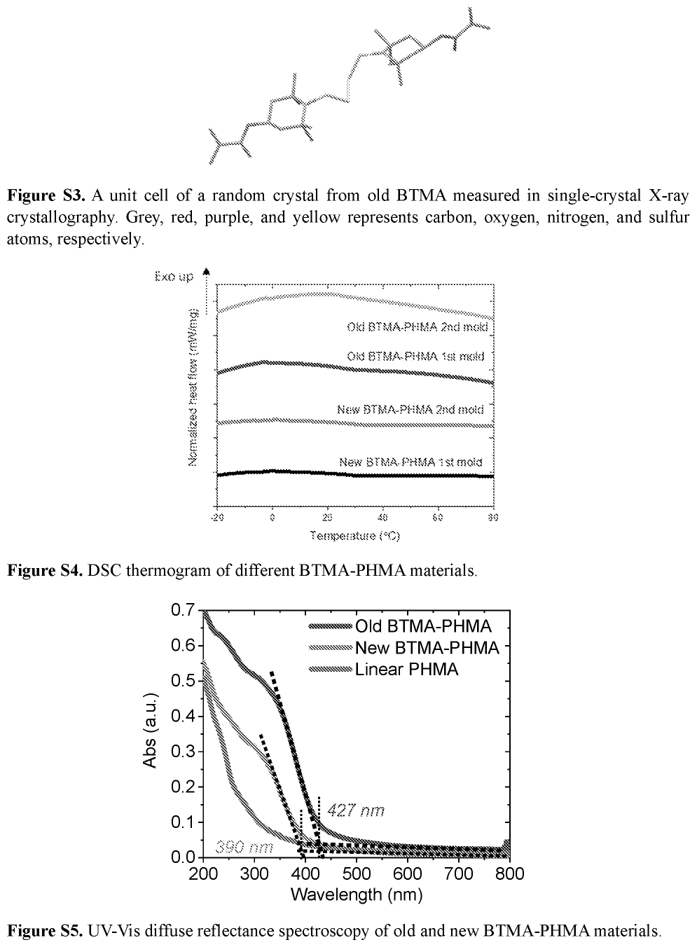

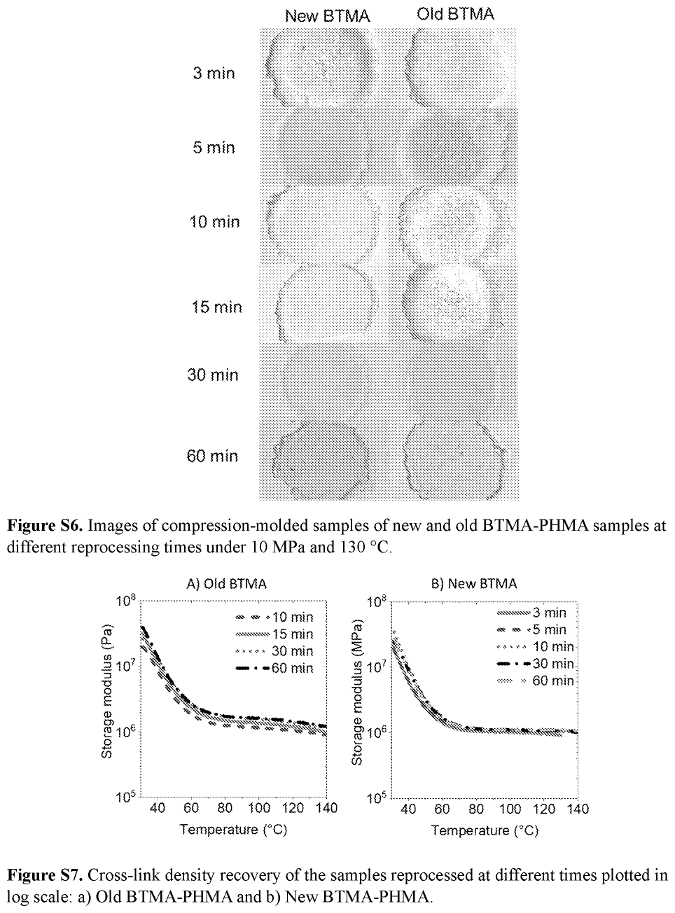

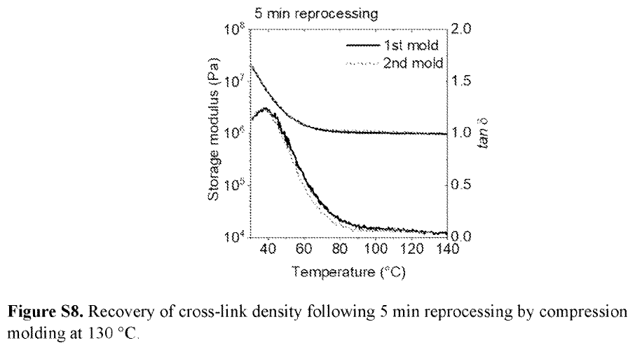

Illustrative polymerizable alkylamines include 2,2,6,6-tetramethyl-4-piperidyl acrylate; 2,2,6,6-tetramethyl-4-piperidyl methacrylate; 2-(tert-butylamino)ethyl acrylate; 2-(tert-butylamino)ethyl methacrylate; 2-(n-butylamino)ethyl methacrylate, 2-(phenylamino)ethyl methacrylate; 2-(cyclohexylamino)ethyl methacrylate; 2,2,6,6-tetramethyl-4-piperidone; 2-methyl-1-(piperazin-1-yl)prop-2-en-1-one; 2-(n-butylamino)ethyl acrylate; 2-(phenylamino)ethyl acrylate; 2-(cyclohexylamino)ethyl acrylate; and 1-(piperazin-1-yl)prop-2-en-1-one. (See FIG. 7.)

A single species of polymerizable alkylamine may be used in the present methods or multiple, different species may be used.

The anhydrous source of sulfur may be a sulfur compound, e.g., S₂Cl₂. To ensure the anhydrous nature of the source of sulfur, the source of sulfur may be subjected to a technique (e.g., distillation) to remove water and other impurities therefrom. In embodiments, anhydrous sulfur monochloride is prepared by means of distillation, wherein the dryness thereof is as described herein. Illustrative anhydrous solvents include anhydrous petroleum ether, anhydrous methyl tetrahydrofuran, anhydrous tetrahydrofuran, anhydrous chloroform, anhydrous dimethyl formamide, anhydrous dimethylacetamide, anhydrous acetonitrile, and anhydrous dichloromethane. By way of illustration only, the amount of water in anhydrous methyl tetrahydrofuran may be ≤0.05%; the amount of water in anhydrous tetrahydrofuran may be ≤50 ppm; and the amount of water in anhydrous dichloromethane may be ≤50 ppm. In embodiments, the anhydrous solvent is anhydrous dichloromethane. As demonstrated in the Examples, below, anhydrous dichloromethane significantly improves the yield of the dynamic crosslinker. A single species of anhydrous solvent or multiple, different species may be used. To ensure the anhydrous nature of the atmosphere, the following technique may be used. A flask to which the anhydrous solvent is added may be first dried by pulling a vacuum on the flask and heating the flask with a heat gun. Next, the flask may be purged with dry nitrogen gas. The vacuum/heating with a heating gun and purging with dry nitrogen gas may be repeated for a total of three cycles.

The conditions under which the reaction mixture is formed to produce the reaction product include parameters such as reaction temperature and reaction time, both of which may be adjusted to achieve a desired yield and/or purity of the dialkylamino disulfide dynamic crosslinker in the reaction product. Illustrative conditions are provided in the Example, below.

The chemical reactions taking place using the present methods involve the coupling of two polymerizable alkylamines to one another via covalent disulfide linkages. By “disulfide linkage” it is meant —(S)₂—, wherein “-” represents a covalent bond to respective nitrogen atoms of each polymerizable alkylamine. Disulfide linkages are distinguished from sulfur linkages involving more than two sulfur atoms, e.g., (i.e., —(S)ₙ—,) wherein n is greater than 2, e.g., 3 (trisulfide linkages), 4 (tetrasulfide linkages), etc. Thus, the dialkylamino disulfide dynamic crosslinker may be represented by PG-(R₁)(R₂)N—(S)₂—N(R₂)(R₁)-PG, wherein PG, R₁, and R₂, have been defined above. Illustrative dialkylamino disulfide dynamic crosslinkers include bis(2,2,6,6-tetramethyl-4-piperidyl acrylate) disulfide; bis(2,2,6,6-tetramethyl-4-piperidyl methacrylate) disulfide; bis(2-(tert-butylamino)ethyl acrylate) disulfide; and bis(2-(tert-butylamino)ethyl methacrylate) disulfide. Analogous dialkylamine disulfide dynamic crosslinkers may be formed using the other polymerizable alkylamines provided above.

Recovering the reaction product from the reaction mixture may comprise precipitating the reaction product from the reaction mixture. This may be accomplished by combining the reaction mixture with water. The step of recovering may further comprise filtering the precipitated reaction product and washing the filtered reaction product as described in the Example, below.

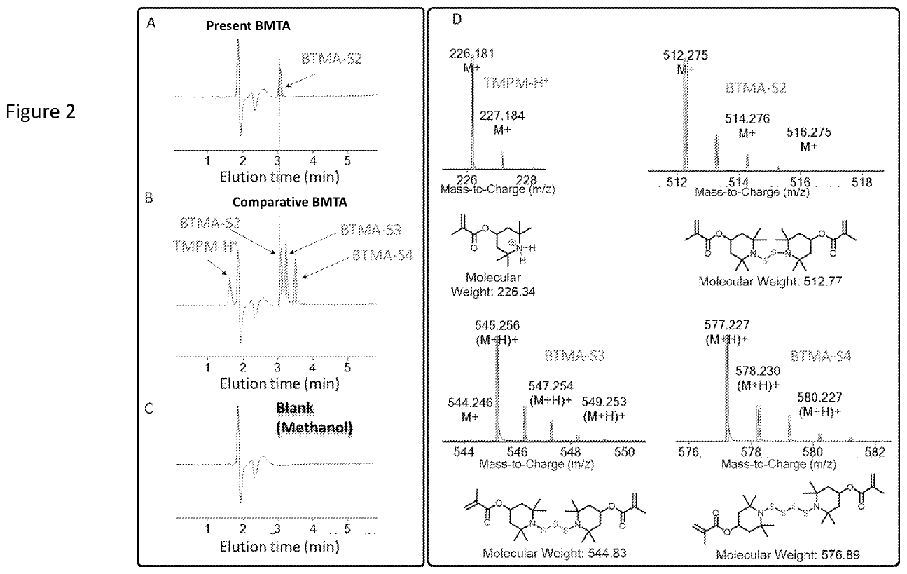

The present methods are characterized by an ability to provide the dialkylamino disulfide dynamic crosslinker described above in the reaction product at high yield and high purity. Regarding yield, this refers to the [(amount of the dialkylamino disulfide dynamic crosslinker in the reaction product)/(theoretical amount of the dialkylamino disulfide dynamic crosslinker in the reaction product based on amount of reactants used)]*100. The yield may be at least 70%, at least 80%, at least 90%, at least 95%, at least 98%, or at least 99%. Regarding purity, this refers to the [(amount of the dialkylamino disulfide dynamic crosslinker in the reaction product)/(amount of reaction product)]*100. The purity may be at least 95%, at least 98%, or at least 99%. Yield and purity may be quantified using liquid chromatography mass spectrometry (LCMS) as described in the Example, below. The methods may be characterized as providing a reaction product that is free of compounds having —(S)ₙ— linkages, wherein n is greater than 2, e.g., 3 (trisulfide linkages), 4 (tetrasulfide linkages), etc. This includes providing a reaction product containing only (i.e., consisting of) the dialkylamino disulfide dynamic crosslinker. Due to the inherent nature of chemical synthesis, terms such as “free,” “consisting of,” and the like do not require that the disulfide dynamic crosslinker be produced with perfect purity. For example, trisulfides and/or tetrasulfides may be present but at an amount of less than 1 weight %. Moreover, as shown in FIG. 2A, the purity achieved by the present methods is essentially 100% as evidenced by a single peak in the liquid chromatograph as measured from the reaction product, the single peak corresponding to the dialkylamino disulfide dynamic crosslinker. By contrast, as shown in FIG. 2B, the reaction product produced using the comparative synthetic technique includes disulfide, trisulfide, and tetrasulfide compounds at approximately the same amounts. The purity of the reaction product produced by the present methods may be further confirmed using single-crystal X-ray crystallography as described in the Example, below.

The reaction products synthesized using the present methods may be used to form dynamic crosslinked polymer networks. As noted above, this reaction product comprises (or consists of) any of the disclosed dialkylamino disulfide dynamic crosslinkers. The dynamic crosslinked polymer network may be formed from a composition comprising (or consisting of) the reaction product; a polymer comprising a carbon-carbon double bond capable of undergoing free radical polymerization, a monomer comprising a carbon-carbon double bond capable of undergoing free radical, or both; and a free radical initiator.

Various polymers and monomers may be used. Vinyl monomers may be used, including styrene, ethylene, 1-octene, vinyl pyridine, an acrylate, a methacrylate, acrylonitrile, vinyl acetate, vinyl chloride, isoprene. Combinations of different monomers may be used. Polymers which may be used include, for example, polybutadiene and polyisoprene. The polymer may be a homopolymer or a co-polymer, e.g., copolymers containing butadiene such as styrene-butadiene block copolymers, random (statistical) copolymers, gradient copolymers, graft copolymers, etc. may be used. Copolymers containing isoprene such as styrene-isoprene block copolymers, random (statistical) copolymers, gradient copolymers, graft copolymers, etc. may also be used. Combinations of different polymers may be used.

Forming the dynamic crosslinked polymer network involves generating free radicals from the free radical initiator present in the composition. These free radicals attack the any of the carbon-carbon double bonds described above, e.g., those present in the monomers, polymers, and the dialkylamino disulfide dynamic crosslinker in the reaction product. In the case of monomers, this results in chain propagation to form polymer chains (e.g., acrylate monomers polymerized to form polyacrylate chains). During this process, the dialkylamino disulfide dynamic crosslinker becomes incorporated into the polymer chains via one of its carbon-carbon double bonds. Since the dialkylamino disulfide dynamic crosslinker is bifunctional, polymer chains (or different portions of an individual polymer chain) become covalently linked together via the dialkylamino disulfide linkages, thereby forming the network. In the case of polymers, similar incorporation and crosslinking occur to form the network, but without the need for chain propagation.

A variety of free radical initiators may be used. Illustrative free radical initiators include azo initiators such as azo nitriles, e.g., 2,2′-Azobis(4-methoxy-2,4-dimethylvaleronitrile) (V-70). Azobisisobutyronitrile is another free radical initiator which may be used. The free radical initiator may be present in the composition at various amounts, e.g., an amount in a range of from 0.001 mol % to 10 mol % (mol % refers to the (moles of initiator)/(total moles of monomer units and dialkylamino disulfide dynamic crosslinker)*100).

Similarly, the dialkylamino disulfide dynamic crosslinker may be present in the composition at various amounts. Illustrative amounts include at least 3 mol %, at least 4 mol %, at least 5 mol %, or in a range of from 3 mol % to 10 mol % (mol % refers to the (moles of dialkylamino disulfide dynamic crosslinker)/(total moles of monomer units and dialkylamino disulfide dynamic crosslinker)*100).

The composition used to form the dynamic crosslinked polymer network may comprise other components, e.g., solvent selected to solubilize/dissolve the dialkylamino disulfide dynamic crosslinker and the monomer/polymer. Other additives may be included, e.g., depending upon desired properties for the network.

The conditions under which the dynamic crosslinked polymer network is formed from the composition include the temperature. Low temperatures may be used to prevent dissociation of the dialkylamino disulfide linkages. This includes temperatures in the range of from 20° C. to 30° C. However, higher temperatures may be used, e.g., up to 150° C. The conditions may also involve use of inert gas (e.g., N₂). These conditions may be applied for a period of time sufficient to achieve full (within 10%, within 5%, within 2%) crosslinking density. Illustrative times include 1 hour, 2 hours, 10 hours, 24 hours. The degree of crosslinking may be determined using dynamical mechanical analysis (DMA) as described in the Example, below.

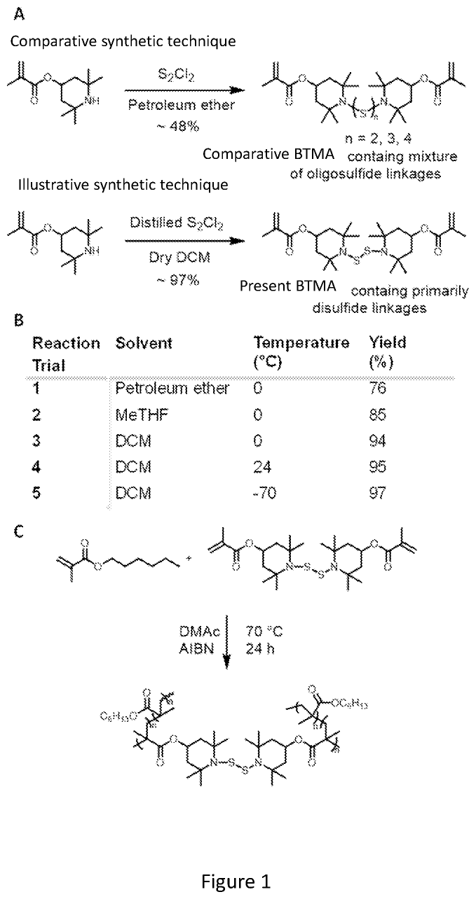

The resulting dynamic crosslinked polymer network is characterized polymer chains covalently linked via the dialkylamino disulfide linkages provided by the dialkylamino disulfide dynamic crosslinker. The crosslinked polymer chains in the network may be represented by (polymer)-(R₁)(R₂)N—(S)₂—N(R₂)(R₁)-(polymer), wherein “polymer” represents different polymer chains or portions of an individual polymer chain and wherein R₁, and R₂, have been defined above. The polymer chain(s) (polymer) may be the polymerization product of any of the carbon-carbon double-bond containing monomers described above (e.g., polyacrylates) or any of the carbon-carbon double-bond containing polymers described above (e.g., polyisoprenes). The chemical structure of an illustrative network is shown in FIG. 1C. The network was formed using the reaction product synthesized according to “Illustrative synthetic technique” shown in FIG. 1A, which provides bis(2,2,6,6-tetramethyl-4-piperidyl methacrylate) disulfide at extremely high purity. This is by contrast to networks formed using reaction products synthesized according to “Comparative synthetic technique” shown in FIG. 1B, which provides about equal amounts of the disulfide, trisulfide, and tetrasulfide compounds.

Because the present methods produce the dialkylamino disulfide dynamic crosslinkers at very high purities, compositions comprising the crosslinkers may also be characterized by high purity. This includes the compositions for forming the dynamic crosslinked polymer networks and the networks themselves. For example, these compositions may be characterized as being free of —(S)ₙ— linkages, wherein n is greater than 2, e.g., 3 (trisulfide linkages), 4 (tetrasulfide linkages), etc. Regarding the dynamic crosslinked polymer networks, when crosslinked, the crosslinks may consist of those provided by the dialkylamino disulfide dynamic crosslinkers, i.e., disulfide crosslinks. Here, terms such as “free,” “consisting of,” and the like have meanings analogous to the definitions provided above.

The dynamic crosslinked polymer networks formed from reaction products synthesized by the present methods may be reprocessed by heating them from a temperature at which dissociation of the dialkylamino disulfide linkages is inactive or substantially inactive, such as room temperature, to an elevated temperature at which the dissociation is activated or significantly enhanced. Reprocessing may involve reshaping (e.g., remolding) at the elevated temperature followed by cooling the network, e.g., to room temperature. During cooling, the dialkylamino disulfide linkages recombine, thereby reforming the network. A single reprocessing cycle refers to a single round of heating, reshaping, and cooling. A single reprocessing cycle may comprise heating to 130° C. under 10 MPa ram force for 1 hour, followed by cooling to room temperature, as described in the Example, below. Reprocessed networks are characterized by full recovery of crosslinking density (as compared to the initial network prior to any reprocessing), which may be evidenced by measuring tensile storage modulus E′ values DMA as described in the Example below. Full recovery means that the E′ values for the reprocessed network are the same (within error) of the initial network prior to any reprocessing. Full recovery of crosslinking density may be obtained after one, two, or more cycles of reprocessing.

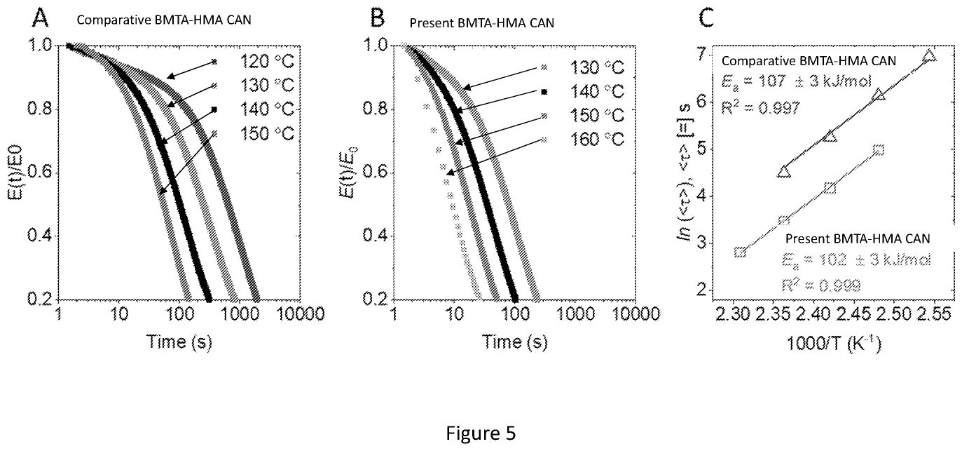

The dynamic crosslinked polymer networks formed from reaction products synthesized by the present methods are characterized by fast average stress relaxation times <t> which may be measured using the technique described in the Example, below. This includes the networks exhibiting a <t> of no more than 200 s, no more than 175 s, or no more than 150 s. (See FIG. 5B.) These values are substantially faster than those exhibited by dynamic crosslinked polymer networks formed from reaction products synthesized using the comparative synthetic technique. (See FIG. 5A.)

The dynamic crosslinked polymer networks formed from reaction products synthesized by the present methods are characterized by a lack of a visible yellow color. This may be quantified using UV-Vis diffuse reflectance spectroscopy as described in the Example, below. This includes the networks exhibiting an onset wavelength of no more than 405 nm, no more than 400 nm, or no more than 390 nm. By contrast, dynamic crosslinked polymer networks formed from reaction products synthesized using the comparative synthetic technique appear yellow and exhibit significantly higher onset wavelengths.

The reaction products synthesized by the present methods are also encompassed by the present disclosure. This includes compositions comprising the reaction products, including the compositions for forming the dynamic crosslinked polymer networks and the networks themselves.

### EXAMPLES

### Experimental

### Materials

All chemicals were commercially available and used as received unless otherwise noted. 2,2,6,6-Tetramethyl-4-piperidyl methacrylate (TMPM) was from TCI America. Anhydrous dichloromethane (DCM), anhydrous methyl tetrahydrofuran (MeTHF), anhydrous petroleum ether, and methanol (99.9%) were from Fischer Scientific. Sulfur monochloride (98%), n-hexyl methacrylate (HMA, 98%), N,N-dimethylacetamide (DMAc, anhydrous, 99.8%), toluene (99.9%) and chloroform-d (99.8 atom % D) were from Sigma-Aldrich. HMA monomer was de-inhibited using inhibitor remover (Sigma Aldrich, 311340) in the presence of calcium hydride (Sigma Aldrich, 90%).

### Synthesis—Comparative Procedure for bis(2,2,6,6-tetramethyl-4-piperidyl methacrylate) disulfide

Bis(2,2,6,6-tetramethyl-4-piperidyl methacrylate) disulfide was prepared as follows. 2,2,6,6-tetramethyl-4-piperidyl methacrylate (8.81 g, 39.09 mmol) was dissolved in a pre-dried petroleum ether (˜90 ml). The solution was then cooled to −70° C. using dry ice and an acetone bath, and then sulfur monochloride (1.31 g, 9.72 mmol) dissolved in pre-dried petroleum ether (1.30 ml) was added dropwise to the solution with continuous stirring. The solution was stirred at −70° C. for 15 min and then at room temperature for an additional 30 min. The reaction mixture was then poured into distilled water and stirred at room temperature overnight. The formed precipitates were filtered off, washed with distilled water, and dried in a vacuum oven at 40° C. for 24 h to give BiTEMPS methacrylate (2.35 g, 48% yield).

### Synthesis—Present Procedure for bis(2,2,6,6-tetramethyl-4-piperidyl methacrylate) disulfide

The present procedure for bis(2,2,6,6-tetramethyl-4-piperidyl methacrylate) disulfide includes certain modifications to the procedure described above. First, sulfur monochloride was distilled under vacuum at room temperature using a kiigelrohr distillation apparatus and stored in a septum-sealed vial under a nitrogen environment. Next, 2,2,6,6-tetramethyl-4-piperidyl methacrylate (TMPM) (7.50 g, 16.66 mmol) was taken in a dry and septum-sealed round-bottom flask fitted with a nitrogen balloon. Then, a dry solvent of choice (˜50 ml) was added to dissolve the TMPM. The solution was then cooled to a desired temperature if needed, and then sulfur monochloride (1.00 g, 3.70 mmol) was dissolved in the same reaction solvent (˜10 ml) and added dropwise to the solution with continuous stirring (see FIG. 1 for solvent and reaction conditions). The solution was stirred at the previously determined temperature for 3 h and then at room temperature for an additional 30 min. The reaction mixture was then poured into distilled water and stirred at room temperature overnight. In the case of the use of MeTHF as a solvent, the reaction mixture was evaporated to dryness in a rotary evaporator, and DCM was added to it. The crude reaction mixture was then poured into distilled water (˜250 ml) and stirred vigorously overnight. The formed precipitates were filtered off, washed with distilled water two times, and dried in a vacuum oven at 50° C. overnight to give BTMA. This dried BTMA was analyzed by ¹H NMR spectroscopy, LCMS, and other analytical techniques.

### Synthesis—BTMA-Containing Networks

The BTMA-HMA networks with 5 mol % BTMA were prepared using AIBN as a radical initiator and performing free-radical polymerization at 70° C. overnight to achieve full conversion.

### Molding and Reprocessing of Networks

Networks were cut into millimeter-sized pieces and processed using a PHI press (Model 0230C-X1). To mold the samples used in dynamic mechanical analysis and stress relaxation experiments, network pieces were hot pressed into ˜1 mm-thick films at 130° C. with a 10-ton ram force for 60 min. After molding, samples were cooled to room temperature in a cold compression mold with a 4-ton ram force for 5 min. Such films are considered to be the 1st molded sample. 2nd molded samples were prepared in a similar way where a 1st molded sample was cut into small pieces and pressed again to obtain 2nd molded sample. In cases where remolding speed was being assessed, the reprocessing continued to be done at 130° C. with a 10-ton ram force but for shorter time scales to assess the shortest reprocessing time at which cross-link density could be fully recovered.

### Dynamic Mechanical Analysis (DMA)

A TA Instruments RSA-G2 Solids Analyzer was used to measure the tensile storage modulus (E′), tensile loss modulus (E″), and the damping ratio (tan δ=E″/E′) of the samples as functions of temperature upon heating rectangular specimens (8 mm×3 mm×1 mm) from −30° C. to 150° C. with a heating rate of 3° C./min under nitrogen atmosphere. The instrument was operated in tension mode at a frequency of 1.00 Hz and 0.03% oscillatory strain. Reported values are the averages of three measurements.

### NMR Spectroscopy

¹H NMR spectroscopy was performed at room temperature using a Bruker Avance III 500 MHz NMR spectrometer. Deuterated chloroform (CDCl₃) was used as a solvent, and the spectrum was reported relative to tetramethylsilane.

### Liquid Chromatography Mass Spectrometry (LC-MS)

Diluted samples were injected on a 1290 Infinity II UHPLC System (Agilent Technologies Inc., Santa Clara, California, USA) onto an Acquity Premier C18 column (1.9 μm, 150×2.1 mm) (Waters Corporation, Milford, Massachusetts, USA) for reversed-phase chromatography which was maintained at 50° C. with a constant flow rate at 0.200 ml/min, using a gradient of mobile phase A (9:1 water/methanol, 10 mM ammonium acetate, 0.2 mM ammonium fluoride) and mobile phase B (2:3:5 acetonitrile/methanol/isopropanol, 10 mM ammonium acetate, 0.2 mM ammonium fluoride). The gradient was programmed as follows: 0-1 min, 95% B; 1-3 min, 95-96% B; 3-6 min, 96-100% B; 6-10 min, hold 100% B; 10-10.10 min, 100-95% B; 10.10-13 min, hold 95% B. After C18 chromatography, the eluents passed through a 1260 Infinity II multi-wavelength detector (Agilent Technologies Inc., Santa Clara, California, USA), prior to MS detection. The absorbance wavelength was set at 214 nm with a 10 nm bandwidth. “MS-Only,” positive ion mode acquisition was conducted on the samples on an Agilent 6545 quadrupole time-of-flight mass spectrometer (Q-TOF LC-MS) equipped with a JetStream ionization source. The source conditions were as follows: gas temperature, 200° C.; drying gas flow, 12 L/min; nebulizer, 50 psi; sheath gas temperature, 300° C.; sheath gas flow, 12 L/min; VCap, 2500 V; fragmentor, 120 V; skimmer, 65 V; and oct 1 RF, 750 V. The acquisition rate in MS-Only mode was 5 spectra/sec between 40-1700 m/z range. The reference ion mass solution was introduced in the ion source using a separate quaternary HPLC pump containing m/z 121.050873 and m/z 922.009798 as reference masses in positive ion mode. Agilent (.d) files were directly imported to MassHunter Qualitative Analysis software (v. 10) where peak areas and mass spectra were extracted for each dataset.

### Differential Scanning Calorimetry (DSC)

A Mettler Toledo DSC822e was used to characterize the glass transition temperatures. Samples were first heated to 150° C. at a rate of 20° C./min and maintained for 5 min, followed by cooling to −60° C. at a rate of −20° C./min. The T_{g} values were obtained from a second heating ramp from −60° C. to 150° C. at a 10° C./min rate using the one-half ΔCₚ method.

### Thermogravimetric Analysis (TGA)

TGA was performed using a Mettler Toledo TGA/DSC3+. Samples were heated under an airflow of 50 min/ml from 25° C. to 800° C. at a rate of 20° C./min. The weight change was recorded as a function of temperature.

### Stress Relaxation

Tensile stress relaxation was characterized using a TA Instruments RSA-G2 Solids Analyzer. Rectangular specimens were mounted on the fixture and allowed to equilibrate at the test temperature for 10 min before starting the test. Once thermal equilibrium was reached, samples were subjected to an instantaneous 5% strain, which was maintained throughout the test. The stress relaxation modulus was recorded until it had relaxed to 20% of its initial value.

### X-Ray Crystallography

Single crystals of C₂₆H₄₄N₂O₄S₂(BTMA) were produced using methanol. A suitable crystal was selected, and the crystal was mounted on a MITIGEN holder in paratone oil on a XtaLAB Synergy R, DW system, HyPix diffractometer. The crystal was maintained at 100.00(11) K during data collection. Using Olex2,³⁵ the structure was solved with the SHELXT³⁶ structure solution program using Intrinsic Phasing and refined with the XL³⁷ refinement package using least squares minimization.

### Crystal Structure Determination of Present BTMA

Crystal Data for C₂₆H₄₄N₂O₄S₂ (M=512.75): monoclinic, space group P21/c (no. 14), a=12.9420(2) Å, b=31.1363(6) Å, c=14.2239(2) Å, β=90.9430(10)°, V=5730.97(16) Å3, Z=8, T=100.00(11) K, μ(Cu Kα)=1.936 mm-1, D_{calc}=1.189 g/mm³, 82193 reflections measured (5.676≤2Θ≤152.092), 11460 unique (Rᵢₙₜ=0.0802, R_{sigma}=0.0373) which were used in all calculations. The final R1 was 0.0681 (I>2σ(I)) and wR2 was 0.1599 (all data).

### Crystal Structure Determination of Comparative BTMA

Crystal Data for C₂₆H₄₄N₂O₄S₄ (M=576.87): monoclinic, space group P21/c (no. 14), a=13.9546(4) Å, b=20.1055(6) Å, c=11.0995(3) Å, β=101.725(3)°, V=3049.14(15) Å3, Z=4, T=99.99(13) K, μ(Cu Kα)=3.123 mm-1, D_{calc}=1.257 g/mm3, 58595 reflections measured (6.468≤2Θ≤153.326), 6266 unique (Rᵢₙₜ=0.0701, R_{sigma}=0.0319) which were used in all calculations. The final R1 was 0.0497 (I>2σ(I)) and wR2 was 0.1382 (all data).

### UV-Vis Diffuse Reflectance Spectroscopy

The UV-Vis-NIR spectrum in diffuse reflectance mode was collected on lightly grounded powder using a Cary 5000 UV-Vis-NIR double-beam spectrophotometer with a monochromator. BaSO₄ powder was used for the baseline collection, and a mixture of sample powder with BaSO₄ was used for the data collection at room temperature. Absorbance data was converted from reflectance data in software using the Kubelka-Munk equation, α/S=(1−R)²/(2R), where α and S are the absorption and scattering coefficients, respectively, and R is the reflectance.

### Results and Discussion

First, the constituents of the comparative BTMA cross-linker were determined. Comparative BTMA was synthesized and characterized it by LCMS, which displayed three peaks for BTMA consistent with BTMA-S2, BTMA-S3, and BTMA-S4 molecules which correspond to disulfide-, trisulfide-, and tetrasulfide-containing versions of BTMA, respectively (see FIG. 2). Additionally, the LC analysis also showed trace levels of various unidentified impurities. This comparative BTMA mixture was crystallized from methanol, and single-crystal X-ray crystallography of a random crystal confirmed the presence of BTMA-S4 molecules (see FIG. S3). We hypothesize that the source of the trisulfide and tetrasulfide linkages is the impure commercial S₂Cl₂, as indicated by a reddish color which is a sign of the presence of impurities like SCl₂, S₂Cl₂, S₃Cl₂, and S₄Cl₂, that can be formed via the reaction with moisture.³³ To avoid this, S₂Cl₂ was purified by vacuum distillation under a high vacuum at room temperature in a kiigelrohr distillation apparatus. This distillation produced a bright golden color compound which is the color of S₂Cl₂ described in the literature.³³ The distilled S₂Cl₂ was subsequently stored in a septum-sealed vial under an inert N₂ atmosphere and was used for the synthesis of the present BTMA-S2 using the modified procedure.

For the synthesis of present BTMA, TMPM was dissolved in various dry solvents, including petroleum ether, MeTHF, and DCM, in a septum-sealed, dry round-bottom flask fitted with a nitrogen balloon to avoid atmospheric moisture. We note that even purified S₂Cl₂ can transform into a mixture of S₃Cl₂ and S₄Cl₂ in the presence of ambient moisture.³³ Then the purified S₂Cl₂, dissolved in a chosen reaction solvent was added to a solution of TMPM at various temperatures (see results in FIG. 1). DCM was found to be the most suitable solvent to dissolve the TMPM even at −70° C., resulting in the highest isolated yield of BTMA (up to 97%) for any solvent used in this study. The solvent mixtures were then poured into distilled water and stirred vigorously overnight to dissolve the protonated TMPM formed during the reaction (see Scheme S1, below) and the remaining excess TMPM.

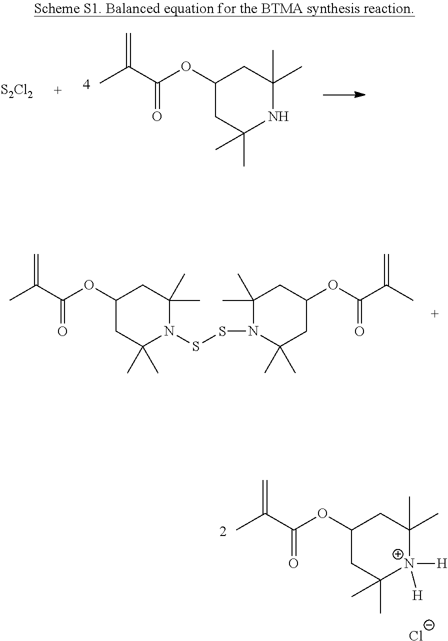

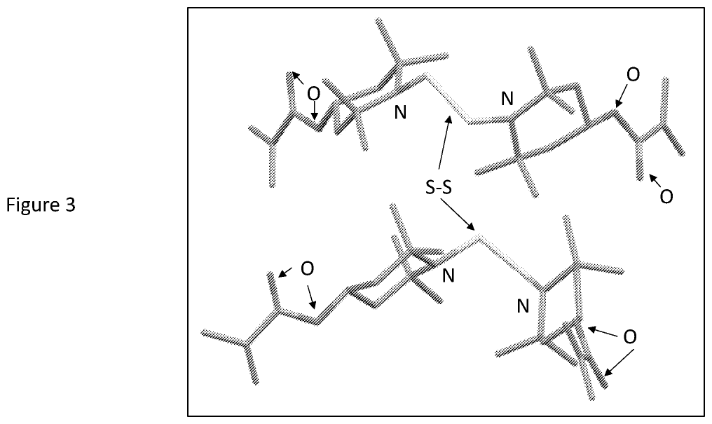

The crude BTMA floated on top of the water phase, which was then vacuum-filtered and washed two times with distilled water. The collected compound was dried in a vacuum oven at 50° C. overnight. The ¹H NMR spectrum (FIG. S1) for the present BTMA was similar to that of the comparative BTMA. To gain further insight, the present BTMA compound was then subjected to LCMS³⁸ which showed primarily a single peak that corresponds to the BTMA-S2 in the mass spectrometry (FIG. 2). Interestingly, this present BTMA is thermally more stable than the comparative BTMA (FIG. S2), with a 1 wt % decomposition temperature (T_{dl %}) of 208° C. for the present BTMA and 188° C. for the comparative BTMA. This compound was then crystallized in methanol and subjected to single-crystal X-ray crystallography which confirms the BTMA-S2 structure (FIG. 3).

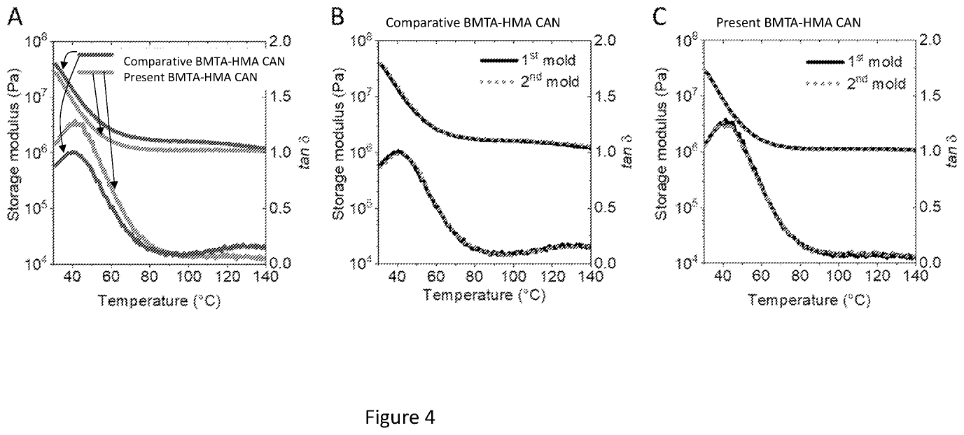

With the structure and composition of the present BTMA having been verified, we compared the dynamic characteristics of BTMA-HMA CANs made with 5 mol % of the present BTMA (BTMA-S2) as compared with 5 mol % of the comparative BTMA. To compare materials based on both BTMAs, HMA was cross-linked with either the comparative or the present BTMA by free-radical polymerization using AIBN at 70° C. to produce comparative BTMA-HMA CANs and present BTMA-HMA CANs. (See FIG. 1.) The polymerization was initiated at 70° C., and gelation was observed after 5 h for both samples. The reaction was continued overnight to ensure complete monomer conversion, within experimental uncertainty. After drying in a vacuum oven at 40° C. for 3 days, the materials were reprocessed at 130° C. under 10 MPa ram force for 1 h, yielding the 1st processed sample. The excellent overlap of the quasi-rubbery plateau values (at temperatures exceeding ˜70° C. in FIG. 4) of tensile storage modulus for 1^{st} and 2^{nd} molds along with Flory's theory of ideal rubber elasticity³⁹ indicate that the cross-link densities of compression molded samples of both comparative BTMA-HMA CANs and present BTMA-HMA CANs remain unaffected by reprocessing at the described conditions, i.e., both comparative BTMA-HMA CANs and present BTMA-HMA CANs can be successfully reprocessed without loss of cross-link density.

It is important to note that samples of the comparative BTMA-HMA CANs had a discernible yellow tint compared to samples of the present BTMA-HMA CANs. To assess that spectroscopically, samples were subjected to UV-Vis diffuse reflectance spectroscopy, which showed that comparative BTMA-HMA CAN absorbs at higher wavelengths (onset wavelength: 427 nm) than present BTMA-HMA CAN (onset wavelength: 390 nm) (see FIG. S5). According to the Kubelka-Munk theory,⁴⁰ materials with a darker or deeper color absorb at longer wavelengths, consistent with the higher coloration in the comparative BTMA-HMA CAN.

DSC analysis showed that both comparative BTMA-HMA CAN and present BTMA-HMA CAN materials have similar T_{g} values of ˜17° C. (See FIG. S4.) Notably, temperature-sweep DMA analysis indicated that, at a given temperature in the quasi-rubbery plateau region, the tensile storage modulus for the present BTMA-HMA CAN is ˜80% as large as that for the old BTMA-HMA CAN. (See FIG. 4, and Table S1, below.)

TABLE S1
Storage modulus (E′) as a function of
T for reprocessed old and new BTMA samples.

| E′ (MPa) | 90° C. | 100° C. | 110° C. | 120° C. | 130° C. | 140° C. |
|---|---|---|---|---|---|---|
| Old BTMA | 1.66 ± | 1.62 ± | 1.56 ± | 1.48 ± | 1.33 ± | 1.23 ± |
| (1^{st} mold) | 0.12 | 0.04 | 0.06 | 0.03 | 0.07 | 0.10 |
| Old BTMA | 1.68 ± | 1.66 ± | 1.57 ± | 1.52 ± | 1.38 ± | 1.28 ± |
| (2^{nd} mold) | 0.12 | 0.07 | 0.03 | 0.12 | 0.09 | 0.09 |
| New BTMA | 1.11 ± | 1.11 ± | 1.12 ± | 1.12 ± | 1.10 ± | 1.08 ± |
| (1^{st} mold) | 0.05 | 0.04 | 0.08 | 0.09 | 0.06 | 0.10 |
| New BTMA | 1.09 ± | 1.10 ± | 1.10 ± | 1.11 ± | 1.10 ± | 1.07 ± |
| (2^{nd} mold) | 0.06 | 0.11 | 0.14 | 0.09 | 0.03 | 0.10 |

Interestingly, as seen in FIG. 4A, the quasi-rubbery plateau tensile storage modulus of the comparative BTMA-HMA CAN decreases somewhat more sharply with increasing temperature than that of the present BTMA-HMA CAN. This difference can be attributed to differences in the temperature sensitivity of the extent of the dissociation between the present BTMA (BTMA-S2) and the comparative BTMA containing oligodisulfides. Nevertheless, in both the comparative CANs and present CANs, the tensile storage modulus values of the 2^{nd} mold materials are, within error, equal to those of the 1^{st} mold materials, indicating full recovery of cross-link density after reprocessing of both CANs.

To compare the dynamic characteristics of the BTMAs, both the comparative BTMA-HMA CAN and the present BTMA-HMA CAN were subjected to stress relaxation experiments (FIG. 5). These curves were fitted with the Kohlrausch-Williams-Watts (KWW) stretched exponential decay function, which generally, with a rare exception, has been found to provide a much better fit for CANs than the single-exponential-decay Maxwell model.^{41,42} At a given temperature, the stress relaxation was substantially faster in the present BTMA-HMA CANs as compared with the comparative BTMA-HMA CANs. For example, at 130° C. the average stress relaxation time, <τ>, for the present BTMA-HMA CAN is only 146 s but is a factor of ˜4 higher at 514 s for the comparative BTMA-HMA CAN (see Table S2, below).

TABLE S2
Characteristic relaxation times (τ*), stretching exponent
(β), average relaxation times (<t>), and Arrhenius activation
energy (Eₐ) values from stress relaxation measurements of
new and old BTMA-PHMA materials at different temperatures

| | T | τ* | | <τ> | | | |
|---|---|---|---|---|---|---|---|
| Sample | (° C.) | (s) | β | (s) | R² | Eₐ (kJ/mol) | R² |
| New BTMA | 130 | 128 | 0.79 | 146 | 0.999 | 102 ± 4 | 0.999 |
| | 140 | 57 | 0.79 | 65 | 0.989 | | |
| | 150 | 29 | 0.82 | 32 | 0.997 | | |
| | 160 | 15 | 0.81 | 16 | 0.998 | | |
| Old BTMA | 120 | 1090 | 0.72 | 1350 | 0.999 | 107 ± 3 | 0.997 |
| | 130 | 422 | 0.73 | 514 | 0.998 | | |
| | 140 | 168 | 0.79 | 192 | 0.997 | | |
| | 150 | 62 | 0.91 | 67 | 0.997 | | |

The dramatic difference in properties can be attributed to the fact that the present BTMA has essentially exclusively disulfide linkages whereas the comparative BTMA contains a mixture of disulfide, trisulfide, and tetrasulfide linkages. Although <τ> at a given temperature is much smaller when present BTMA is used versus comparative BTMA, the apparent activation energies are the same within experimental uncertainty for the CANS made with present BTMA (107±3 kJ/mol) and those made with comparative BTMA (102±3 kJ/mol). Additionally, both values are in good accordance with a value reported by Torkelson and coworkers in ref. 27 for comparative BTMA-HMA CANs.

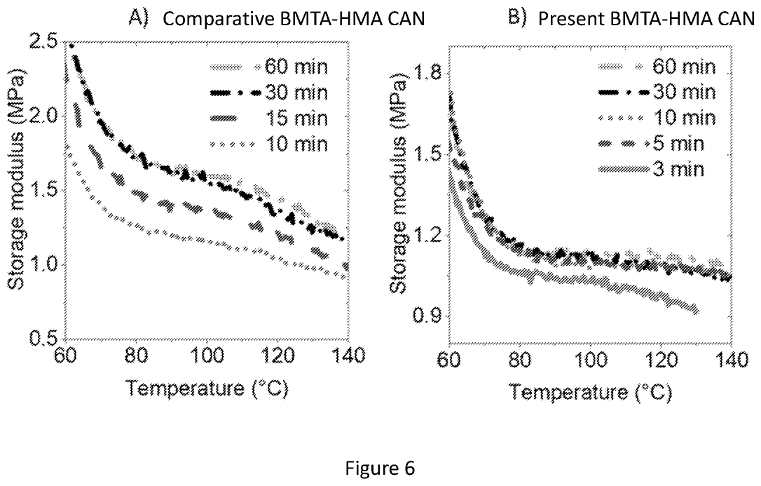

Other things being equal in CANs, faster stress relaxation times are indicative of faster dynamic chemistry which, in turn, should be correlated with faster reprocessability. Here, we observed that compression molding at 130° C. with 10-ton ram force of the present BTMA-HMA CANs required only 5 min of reprocessing time to achieve recovery of rubbery plateau modulus and thus cross-link density. In contrast, using the same compression molding temperature and 10-ton ram force, the comparative BTMA-HMA CANs required 20 min of reprocessing by compression molding time to recover the same cross-link densities as in the original molding (see FIG. 6, FIGS. S6, S7, and S8). The 5 min compression molding time frame to achieve reprocessing with full cross-link density recovery makes the present BTMA-HMA CAN among the fastest CAN materials ever to be reprocessed.⁴³⁻⁴⁵ Such fast processability for these CAN materials portend opportunities for realistic processing and reprocessing by melt-extrusion, an important polymer processing method for industrial applications.

### CONCLUSIONS

Using a modified synthetic procedure, we prepared a BTMA-dynamic cross-linker with a higher isolated yield of up to 97% compared to BTMA prepared using an existing synthetic technique (isolated yield of ˜48%). LCMS characterization showed that the comparative BTMA contained substantial levels of disulfide, trisulfide, and tetrasulfide linkages in addition to some other traces of unidentified impurities. In contrast, LCMS and single-crystal X-ray crystallography showed present BTMA was highly pure and consisted nearly exclusively of disulfide linkages (BTMA-S2). Both present BTMA and comparative BTMA produced robust CANs when reacted with n-hexyl methacrylate by free-radical polymerization, and both types of CANs could be reprocessed with full recovery of cross-link density. The CANs made from 5 mol % comparative BTMA or 5 mol % present BTMA possessed similar properties in the crosslinked state but dramatic differences in their dynamic character. Although the materials have similar activation energies for elevated-temperature stress relaxation, at 130° C. the present BTMA-HMA CANs exhibited a factor of 4 shorter average stress relaxation time, i.e., substantially faster stress relaxation, than the comparative BTMA-HMA CANs. This difference is attributed to nearly exclusive disulfide linkages in the present BTMA materials. Such faster stress relaxation times translate into a factor of 4 faster reprocessing of present BTMA-HMA CANs versus comparative BTMA-HMA CANs, with the present BTMA-HMA CANs requiring only 5 min of compression molding at 130° C. and 10-ton ram force to achieve complete cross-link density recovery after reprocessing. Such results demonstrate that CANs made with present BTMA have the potential to be processed and reprocessed by continuous melt-processing operations, e.g., melt extrusion.

### ADDITIONAL EMBODIMENTS

Additional embodiments of this invention are set forth below:

Embodiment 1: A polymerizable composition comprising
a crosslinker composition comprising a disulfide crosslinker having a formula R—R″R′N—S—S—NR′R″—R, wherein each R is a polymerizable group comprising a C═C double bond capable of undergoing a free radical polymerization, and R′ and R″ are each independently an alkyl group or a bivalent alkylene group, wherein the disulfide crosslinker does not comprise a urethane group;
a monomer, polymer, or combination thereof, comprising at least one C═C double bond capable of undergoing a free radical polymerization; and
a free radical initiator;
wherein the purity of the disulfide crosslinker in the crosslinker composition is 90% or higher.

Embodiment 2: The polymerizable composition of Embodiment 1, wherein the purity of the disulfide crosslinker in the crosslinker composition is 95% or higher.

Embodiment 3: The polymerizable composition of Embodiment 1, wherein in the total amount of polysulfide byproduct(s) in the disulfide crosslinker is no more than 10 wt % (such as no more than 5 wt %) of the crosslinker composition, optionally wherein the polysulfide byproduct has the formula R—R″R′N—Sₙ—NR′R″—R, wherein n is 3 or more (e.g., n is 3, 4, or 5).

Embodiment 4: The polymerizable composition of any one of Embodiments 1-3, wherein R′ and R″ are each independently an unsubstituted alkyl group or a bivalent alkylene group.

Embodiment 5: The polymerizable composition of any one of Embodiments 1-3, wherein R′ and R″ are each bound to the same nitrogen and are covalently bound together to form a bivalent nitrogen-containing cycloalkylene group.

Embodiment 6: The polymerizable composition of any one of Embodiments 1-3, wherein the disulfide crosslinker is bis(2,2,6,6-tetramethyl-4-piperidyl) disulfide comprising the two polymerizable groups.

Embodiment 7: The polymerizable composition of any one of Embodiments 1-6, wherein the two polymerizable groups are selected from the group consisting of acrylates, methacrylates, styrene, vinyl pyridine, and combinations thereof.

Embodiment 8: The polymerizable composition of Embodiment 6 or 7, wherein the disulfide crosslinker is bis(2,2,6,6-tetramethyl-4-piperidyl methacrylate) disulfide.

Embodiment 9: The polymerizable composition of any one of Embodiments 1-3, wherein the polymerizable composition comprises a monomer having a formula R₁R₂C═CR₃R₄, wherein each of R₁-R₄ is independently selected from the group consisting of hydrogen, halogen, alkyl, aryl, arylalkyl, alkenyl, arylalkenyl, alkoxycarbonyl, and alkylaminecarbonyl.

Embodiment 10: The polymerizable composition of any one of Embodiments 1-3, wherein the polymerizable composition comprises a monomer selected from the group consisting of an olefin monomer, a diene monomer, an acrylate monomer, a vinyl monomer, and combinations thereof.

Embodiment 11: The polymerizable composition of Embodiment 10, wherein the monomer comprise at least one member selected from the group consisting of ethylene, propylene, 1-butylene, 1-pentene, 1-hexene, 1-heptene, 1-octene, 1-nonene, 1-decene, a vinyl ester (e.g., vinyl acetate), a vinyl halide, a vinyl nitrile, a vinyl silane, a vinyl pyridine, a styrenic monomer, and combinations thereof.

Embodiment 12: The polymerizable composition of Embodiment 10, wherein the monomer is ethylene, or ethylene and vinyl acetate.

Embodiment 13: The polymerizable composition of any one of Embodiments 1-3, wherein the polymerizable composition comprises a polymer comprising repeating units derived from an olefin monomer, a diene monomer, an acrylate monomer, a vinyl monomer, or combinations thereof, wherein the polymer optionally has a molecular weight of from 1×10² g/mol to 1×10⁷ g/mol, measured according to gel permeation chromatography.

Embodiment 14: The polymerizable composition of any one of Embodiments 1-3, wherein the free radical initiator comprises at least one member selected from the group consisting of a peroxide, an azo compound, a peracetate compound, and a nitroxide.

Embodiment 15: A reversibly-crosslinked polymer network comprising the reaction product of the components in the polymerizable composition according to any one of Embodiments 1-14, wherein the reversibly-crosslinked polymer network contains —S—S-linkages covalently linking polymer chains.

Embodiment 16: The reversibly-crosslinked polymer network of Embodiment 15, wherein the crosslinker composition is incorporated into the reversibly-crosslinked polymer network in an amount ranging from about 0.1 mol % to about 30 mol % (such as from about 1 mol % to about 10 mol %, e.g., about 5 mol %), relative to 100 mol % of the total amount of the reversibly-crosslinked polymer network.

Embodiment 17: The reversibly-crosslinked polymer network of Embodiment 15, wherein the reversibly-crosslinked polymer network possesses a faster reprocessing time, characterized by having a stress relaxation at a rate faster than (e.g., at least 50%, 100%, 1.5 times, 2 times, 2.5 times, 3 times, 3.5 times, or 4 times faster than) a reversibly-crosslinked polymer network formed with a crosslinker composition containing a polysulfide crosslinker, having a formula R—R″R′N—Sₙ—NR′R″—R, wherein n is 3 or more (e.g., n is 3, 4, or 5).

Embodiment 18: The reversibly-crosslinked polymer network of Embodiment 15, wherein the reversibly-crosslinked polymer network fully recovers its crosslink density, after reprocessing, at a rate faster than (e.g., at least 50%, 100%, 1.5 times, 2 times, 2.5 times, 3 times, 3.5 times, 4 times, 4.5 times, or 5 times faster than) a reversibly-crosslinked polymer network formed with a crosslinker composition containing a polysulfide crosslinker, having a formula R—R″R′N—Sₙ—NR′R″—R, wherein n is 3 or more (e.g., n is 3, 4, or 5).

Embodiment 19: A method of forming a reversibly-crosslinked polymer network, comprising:
reacting, by free radical polymerization, the components in the polymerizable composition according to any one of Embodiments 1-14, to produce a reversibly-crosslinked polymer network containing —S—S— linkages covalently linking polymer chains.

Embodiment 20: The method of Embodiment 19, further comprising:
reprocessing the reversibly-crosslinked polymer network at a temperature greater than room temperature (such as a temperature of greater than 50° C.) to dissociate the crosslinking bonds of the reversibly-crosslinked polymer.

Embodiment 21: The method of Embodiment 20, wherein the reprocessing step comprises:
heating the reversibly-crosslinked polymer network to a temperature greater than room temperature (such as a temperature of greater than 50° C.) to induce the cleavage of the reversible —S—S— linkages;
reshaping the reversibly-crosslinked polymer network; and
cooling the reversibly-crosslinked polymer network such that the cleavage of the reversible —S—S— linkages is stopped.

Embodiment 22: The method of Embodiment 20 or 21, wherein the reprocessing step (such as the heating step) for the reversibly-crosslinked polymer network occurs at a temperature lower than the temperature for reprocessing a reversibly-crosslinked polymer network formed with a crosslinker composition containing a polysulfide crosslinker, having a formula R—R″R′N—Sₙ—NR′R″—R, wherein n is 3 or more (e.g., n is 3, 4, or 5).

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the invention to enable one skilled in the art to utilize the invention in various embodiments and with various modifications as suited to the particular use contemplated. It is intended that the scope of the disclosure be defined by the claims appended hereto and their equivalents.

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

What is claimed is:

1. A method of synthesizing a dialkylamino disulfide dynamic crosslinker, the method comprising:
(a) forming a reaction mixture by combining a polymerizable alkylamine with an anhydrous source of sulfur in an anhydrous solvent under an anhydrous atmosphere to form a reaction product comprising a dialkylamino disulfide dynamic crosslinker; and
(b) recovering the reaction product from the reaction mixture.

2. The method of claim 1, wherein the polymerizable alkylamine is represented by PG-(R₁)(R₂)NH, wherein PG is a polymerizable group, R₁ and R₂ are each an alkyl group, and “-” is a covalent bond to either R₁ or R₂; or PG is a polymerizable group, R₁ and R₂ are covalently bound together to form a cycloalkyl group containing the nitrogen atom, and “-” is a covalent bond to the cycloalkyl group.

3. The method of claim 2, wherein PG is a (meth)acrylate group.

4. The method of claim 1, wherein the polymerizable alkylamine is selected from 2,2,6,6-tetramethyl-4-piperidyl acrylate; 2,2,6,6-tetramethyl-4-piperidyl methacrylate; 2-(tert-butylamino)ethyl acrylate; 2-(tert-butylamino)ethyl methacrylate; 2-(n-butylamino)ethyl methacrylate, 2-(phenylamino)ethyl methacrylate; 2-(cyclohexylamino)ethyl methacrylate; 2,2,6,6-tetramethyl-4-piperidone; 2-methyl-1-(piperazin-1-yl)prop-2-en-1-one; 2-(n-butylamino)ethyl acrylate; 2-(phenylamino)ethyl acrylate; 2-(cyclohexylamino)ethyl acrylate; 1-(piperazin-1-yl)prop-2-en-1-one; and combinations thereof.

5. The method of claim 1, wherein the anhydrous source of sulfur is anhydrous S₂Cl₂.

6. The method of claim 1, wherein the anhydrous solvent is anhydrous dichloromethane.

7. The method of claim 1, wherein the dialkylamino disulfide dynamic crosslinker in the reaction product has a yield of at least 70%.

8. The method of claim 7, wherein the dialkylamino disulfide dynamic crosslinker in the reaction product has a purity of at least 95%.

9. The method of claim 1, wherein the reaction product is free of a compound having a —(S)ₙ— linkage, wherein n is greater than 2.

10. The method of claim 1, wherein the reaction product consists of the dialkylamino disulfide dynamic crosslinker.

11. A reaction product synthesized according to the method of claim 1, wherein the reaction product comprises the dialkylamino disulfide dynamic crosslinker.

12. A composition comprising a reaction product comprising a dialkylamino disulfide dynamic crosslinker represented by PG-(R₁)(R₂)N—(S)₂—N(R₂)(R₁)-PG,
wherein PG is a polymerizable group, R₁ and R₂ are each an alkyl group, and “-” is a covalent bond to either R₁ or R₂; or PG is a polymerizable group, R₁ and R₂ are covalently bound together to form a cycloalkyl group containing the nitrogen atom, and “-” is a covalent bond to the cycloalkyl group; and
wherein the dialkylamino disulfide dynamic crosslinker in the reaction product has a purity of at least 95%.

13. The composition of claim 12, wherein PG is a (meth)acrylate group.

14. The composition of claim 12, wherein the reaction product and the composition are both free of a compound having a —(S)ₙ— linkage, wherein n is greater than 2.

15. A dynamic crosslinked polymer network comprising polymer chains covalently linked by dialkylamino disulfide linkages formed from a reaction product comprising a dialkylamino disulfide dynamic crosslinker represented by PG-(R₁)(R₂)N—(S)₂—N(R₂)(R₁)-PG,
wherein PG is a polymerizable group, R₁ and R₂ are each an alkyl group, and “-” is a covalent bond to either R₁ or R₂; or PG is a polymerizable group, R₁ and R₂ are covalently bound together to form a cycloalkyl group containing the nitrogen atom, and “-” is a covalent bond to the cycloalkyl group; and
wherein the dialkylamino disulfide dynamic crosslinker in the reaction product has a purity of at least 95%.

16. The dynamic crosslinked polymer network of claim 15, wherein PG is a (meth)acrylate group.

17. The dynamic crosslinked polymer network of claim 15, wherein the wherein the reaction product and the network are both free of a compound having a —(S)ₙ— linkage, wherein n is greater than 2.
